SEQUENCE LISTING (6 sequences)

SEQ ID NO: 1 - DNA, 1632 bp, Ralstonia solanacearum
  misc_feature (1)..(1632): rscK60 cds
atgcatcgccccccgcttgcggtcggacgtttcttttctacattgaagtgcccgctgcacatcgcatcgccgggcttccgggtggctggctttcctgcgccgccccacaccaacaccaacggactcatccaggagaaatcaatggtcgtgcgtagaacggtgctgaaggcaatcgccggaaccagtgtcgccacggtattcgcgggcaagctgaccggtctctccgctattgctgccgatgcggcccccttgctggtacggcgcaacctgcacggcatgaagctggacgacccggacctgtcggcctaccgcgagttcgtcagcatcatgaaaagcaagaaccagacgcagccgctgagctggctcggctacgccaaccagcacggctcgctcaacggtggcttcaagtactgcccgcacggcgattggtacttcctgccctggcaccgcggcttcatgctgatgtacgaacgggccgtggctgcgctcaccggctacaagtcattcgccatgccgtactggaactggaccgaagaccgcctgctgcccgaagccttcaccgccaagacctacaacggcaagccgaacccgctctacgtgcccaaccggaacgatctgaccggtccctatgccctcaccgacgccatcgtcggccagaaggaggtcatggacaagatctacgccgaaaccaactttgaagtcttcggcaccagccgctcggtggatcgctcggtccagccgccgctggtccagaacagcctcgaccccaaatgggtgccgatgggcggcggcacgcagggcattctggaacgcacgccgcacaacaccgtccacaacaacatcggcgccttcatgcccaccgccgcctcgccacgcgacccggtgttcatgatgcaccacggcaacatcgaccgggtgtgggccacctggaatgcgctgggccgcaagaactcgaccgacccgctgtggctgggcatgaagttccccaacaactacattgacccgcagggccggtactacacgcagggcgtgagcgacctgctgagtaccgaggcgctgggctaccgctatgacgtcatgccgcgcgccgacaacaaggtggtgaacaacgcccgcgccgagcacctgctggccctgttcaagaccggcgacagcgtcaagctggctgaccacatgaagctgcgcagcgtgctgaagggggaacatccggccgccacggcagtcctaccgctcaacagcgctgtccagttcgaggctggcaccgtaacgggtgccctaagtgccgacgccgataccggcaagaccaccgaagtcgtggccctgatcaagaacatcaggatgtccgagaacgtgatcagcatccgggtcttcgtcaacctgccggacgccagtctcgatgtgccggaaaccaacccgcacttcgtcaccacgctcagcttcctgacgcacgcggttggacacgaccaccatgccctgccgtcgaccatggtgaacctgaccgacacgctgaaggcgctcaatatccgggacgacaatttctcgatcaacctggttgctgtgcccaagcccggcgtcgccgtggagagcagcggcagcgtgacacccgagtcgatcgaggtcgccgtcatttga

SEQ ID NO: 2 - protein, 543 aa, Ralstonia solanacearum
  PEPTIDE (1)..(543): RscK60-Oxidase
MHRPPLAVGRFFSTLKCPLHIASPGFRVAGFPAPPHTNTNGLIQEKSMVVRRTVLKAIAGTSVATVFAGKLTGLSAIAADAAPLLVRRNLHGMKLDDPDLSAYREFVSIMKSKNQTQPLSWLGYANQHGSLNGGFKYCPHGDWYFLPWHRGFMLMYERAVAALTGYKSFAMPYWNWTEDRLLPEAFTAKTYNGKPNPLYVPNRNDLTGPYALTDAIVGQKEVMDKIYAETNFEVFGTSRSVDRSVQPPLVQNSLDPKWVPMGGGTQGILERTPHNTVHNNIGAFMPTAASPRDPVFMMHHGNIDRVWATWNALGRKNSTDPLWLGMKFPNNYIDPQGRYYTQGVSDLLSTEALGYRYDVMPRADNKVVNNARAEHLLALFKTGDSVKLADHMKLRSVLKGEHPAATAVLPLNSAVQFEAGTVTGALSADADTGKTTEVVALIKNIRMSENVISIRVFVNLPDASLDVPETNPHFVTTLSFLTHAVGHDHHALPSTMVNLTDTLKALNIRDDNFSINLVAVPKPGVAVESSGSVTPESIEVAVI

SEQ ID NO: 3 - protein, 496 aa, Artificial Sequence
  RscK60-del Oxidase
  PEPTIDE (1)..(496): RscK60-del Oxidase
MVVRRTVLKAIAGTSVATVFAGKLTGLSAVAADAAPLRVRRNLHGMKMDDPDLSAYREFVGIMKGKDQTQALSWLGFANQHGTLNGGYKYCPHGDWYFLPWHRGFVLMYERAVAALTGYKTFAMPYWNWTEDRLLPEAFTAKTYNGKTNPLYVPNRNELTGPYALTDAIVGQKEVMDKIYAETNFEVFGTSRSVDRSVRPPLVQNSLDPKWVPMGGGNQGILERTPHNTVHNNIGAFMPTAASPRDPVFMMHHGNIDRVWATWNALGRKNSTDPLWLGMKFPNNYIDPQGRYYTQGVSDLLSTEALGYRYDVMPRADNKVVNNARAEHLLALFKTGDSVKLADHIRLRSVLKGEHPVATAVEPLNSAVQFEAGTVTGALGADVGTGSTTEVVALIKNIRIPYNVISIRVFVNLPNANLDVPETDPHFVTSLSFLTHAAGHDHHALPSTMVNLTDTLKALNIRDDNFSINLVAVPQPGVAVESSGGVTPESIEVAVI

SEQ ID NO: 4 - DNA, 33 bp, Artificial Sequence
  Primer forward
  primer_bind (1)..(33): rsck60-1f
ttaagaattcatgcatcgccccccgcttgcggt

SEQ ID NO: 5 - DNA, 34 bp, Artificial Sequence
  Primer reverse
  primer_bind (1)..(34): rsck60-2r
atataagctttcaaatgacggcgacctcgatcga

SEQ ID NO: 6 - DNA, 34 bp, Artificial Sequence
  Primer forward
  primer_bind (1)..(34): rsck60-3f
ttaagaattcatggtcgtgcgtagaacggtgctg

(19) United States
(12) Patent Application Publication
(10) Pub. No.: US 2024/0200107 A1
(43) Pub. Date: Jun. 20, 2024

(54) METHOD FOR PRODUCING A HYDROXYTYROSOL

(71) Applicant: Wacker Chemie AG, Munich (DE)

(72) Inventor: Rupert PFALLER, Munich (DE)

(73) Assignee: Wacker Chemie AG, Munich (DE)

(21) Appl. No.: 18/277,398

(22) PCT Filed: Mar. 3, 2021
(86) PCT No.: PCT/EP2021/055378

§ 371 Date: Aug. 16, 2023

(51) Int. Cl. C12P 7/22; C12N 9/02; C12N 15/70

(52) U.S. Cl. CPC C12P 7/22; C12N 9/0059; C12N 15/70

(57) ABSTRACT

A process for producing hydroxytyrosol (HTS) by enzymatic conversion of tyrosol to HTS is provided. The process includes a reaction mixture comprising tyrosol, a compound selected from the group consisting of erythorbic acid and erythorbate, and an oxidase with an amino acid sequence selected from the group consisting of SEQ ID NO: 2 and an amino acid sequence homologous with SEQ ID NO: 2. HTS is isolated from the reaction mixture.

The invention relates to a process for producing hydroxytyrosol (HTS) by enzymatic conversion of tyrosol to HTS, characterized in that the reaction mixture comprises i) tyrosol and ii) a compound selected from the group consisting of erythorbic acid and erythorbate and iii) an oxidase with an amino acid sequence selected from the group consisting of SEQ ID NO: 2 and an amino acid sequence homologous to SEQ ID NO: 2, and HTS is isolated from the reaction mixture.

Hydroxytyrosol (HTS: 3,4-dihydroxyphenylethanol: CAS number 10597-60-1) is an effective antioxidant and has been the subject of great interest in recent years owing to its positive effects on health. HTS is considered to be an active component in the Mediterranean diet. The European Food Safety Authority (EFSA) allowed a positive health claim for polyphenols from olives, recommending a daily HTS dose of at least 5 mg. An inflammation-inhibiting effect of HTS has also been described. There also exists studies showing that HTS in vitro has antimicrobial properties against pathogens of the respiratory pathway and the gastrointestinal tract, as against some strains of the Vibrio, Salmonella or Staphylococcus genera, and that the doses used can quite possibly compete with those of antibiotics, e.g. ampicillin. The substance is additionally ascribed a neuroprotective and antiproliferative and proapoptotic effect. These properties mean that HTS is a very interesting and sought-after substance that finds use in pharmaceuticals, food supplements, functional foods or else in cosmetics.

HTS available on the market to date comes mainly from olives, olive leaves or wastewater which is obtained in olive oil production and is supplied in the form of an extract: the proportion of HTS in these products is usually very low. Examples of these are HIDROX® with an HTS content below 12%, or OPEXTAN™, which contains about 4.5% HTS.

As well as the isolating of natural HTS from olives, methods of synthetically producing this substance have been described.

For example, TN SN03042 A1 or the corresponding publication by Allouche et al. (2004), J. Agric. Food Chem. 52: 267-273 describes the extraction and purification of HTS from olive oil wastewater.

A process for chemical preparation of HTS is disclosed, for example, in EP 2 774 909 B1, where the reactant of the general formula (1)

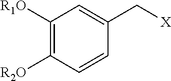

is reacted in the presence of an aluminum compound and an aqueous hydroxycarboxylic acid at a pH<3, and the HTS formed is isolated by extraction.

Also known, in addition, are biotechnology methods for production of HTS. For example, tyrosol can be hydroxylated to give HTS in the presence of atmospheric oxygen according to the formula

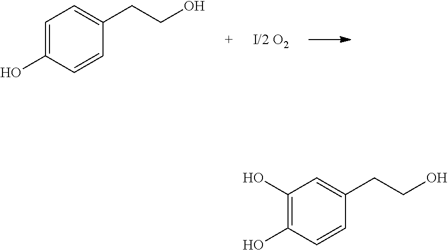 [SMILES: OCCc1ccc(O)cc1 ; OCCc1ccc(O)c(O)c1]

For example, EP 3 234 164 B1 describes a process for enzymatic conversion of tyrosol to HTS with a tyrosinase enzyme from Ralstonia solanacearum or a functional derivative thereof in a reaction mixture with ascorbic acid, where the functional derivative is an engineered variant of the tyrosinase enzyme from R. solanacearum that has 1 to 5 amino acid changes by comparison with the wild-type tyrosinase enzyme, where the or each change is selected from insertion, addition, deletion and substitution of an amino acid.

EP 3 234 164 B1 discloses a tyrosinase, the activity of which is not inhibited by 27.6 g/L (199.7 mM) of the tyrosol substrate, 30.8 g/L (199.7 mM) HTS, and up to a concentration of 0.4 M of the sodium salt of ascorbic acid. In a biotransformation, it was possible to convert 150 mM tyrosol to HTS in the presence of 300 mM of the sodium salt of ascorbic acid (2-fold molar excess over the tyrosol reactant) by means of an engineered variant of R. solanacearum tyrosinase produced by recombinant means in E. coli on a shaken flask scale (laboratory scale) and used in the form of a cell-free lysate. At an enzyme dose of 2 mg/L per 1 mM tyrosol (i.e. a very high dose of 300 mg of enzyme per 150 mM tyrosol) of the tyrosol substrate, the reaction time until complete conversion was about 6 h.

The process disclosed in EP 3 234 164 B1 is therefore inadequate for industrial use. Producibility of the tyrosinase has been disclosed only on a laboratory scale. The amount of the tyrosol reactant used is comparatively low at 150 mM, and it was necessary to produce a cell-free lysate of the tyrosinase-producing cells.

CN 101624607 B discloses a process in which 25 g/L (180 mM) tyrosol is converted to HTS by an oxidase in the presence of 50 g/L (284 mM) ascorbic acid (molar ratio of tyrosol to ascorbic acid: 1:1.57), and HTS is isolated in a multistage process by nanofiltration, column chromatography, extraction and distillation. Under these conditions, HTS was obtained in a purity of 88.3%. HTS in higher purity would be obtained only by complex immobilization of the oxidase on a support and another column chromatography operation using the abovementioned process steps in order to isolate HTS in higher purity.

The process from CN 101624607 B will be considered by the person skilled in the art to have some gaps in the disclosure. The origin of the oxidase enzyme is insufficiently disclosed. There is likewise no information about the production process for the enzyme which is important for economic viability. Moreover, there are no details of the method of determining the product purity.

The process disclosed in CN 101624607 B is insufficiently suitable in turn for industrial use. The amount of the tyrosol reactant used is comparatively low at max. 180 mM (25 g of tyrosol/kg of batch), and a 1.57-fold molar excess of ascorbic acid is very high. In the case of biotransformation with immobilized enzyme, the enzyme first has to be purified in a complex manner, and the use amounts of the tyrosol reactant are then much lower than in the batch with non-immobilized enzyme. Moreover, the HTS has to be worked up in this process over five stages, namely nanofiltration, chromatography, ethyl acetate extraction of the eluate, distillation of the extract, and another column chromatography operation. These cost-determining factors necessitate an improvement in the process disclosed in CN 101624607 B in order to improve economic viability of biotechnological production of HTS.

Li et al. (2018), ACS Synth. Biol. 7: 647-654 describes a route to biosynthetic production of HTS that is not based on biotransformation of tyrosol.

It is an object of the present invention to provide a process improved over the prior art and having higher economic viability, which enables production of hydroxytyrosol in a simple manner and in high purity.

The object is achieved by a process for producing hydroxytyrosol (HTS) by enzymatic conversion of tyrosol to HTS, characterized in that the reaction mixture comprises i) tyrosol and ii) a compound selected from the group consisting of erythorbic acid and erythorbate and iii) an oxidase with an amino acid sequence selected from the group consisting of SEQ ID NO: 2 and an amino acid sequence homologous to SEQ ID NO: 2, and HTS is isolated from the reaction mixture.

Erythorbic acid used in the context of this invention, in general, is preferably D-erythorbic acid. Erythorbate is a salt of erythorbic acid, preferably sodium salt, sodium D-erythorbate, more preferably sodium D-ery thorbate x H₂O. Erythorbic acid or the salt thereof is an auxiliary in the reaction and is also referred to hereinafter by the term “protective substance”. The reason for this term is that, in the presence of the protective substance, HTS is the end product in the oxidation from tyrosol, i.e. HTS is “protected”, whereas, in the absence of protective substance, HTS is oxidized further. A protective substance is defined as a substance that contributes to stabilization of the product.

It is a feature of the oxidase (also referred to hereinafter as protein (iii)) that it
(1) has oxidase activity, i.e. can convert tyrosol even in concentrations of >0.2 M in the presence of atmospheric oxygen, and during this reaction tolerates erythorbic acid or ery thorbate even in high concentrations of >0.4 M and
(2) has the amino acid sequence of SEQ ID NO: 2 (RscK60 oxidase) or an amino acid sequence homologous to SEQ ID NO: 2,
where SEQ ID NO:2 is the protein identified by accession number CCF97399.1 in the NCBI protein database.

What is meant by an amino acid sequence homologous to the sequence annotated as polyphenol oxidase/catechol oxidase and disclosed in SEQ ID NO: 2 is that, over the entire sequence range from amino acid 1 to amino acid 543, there is a sequence identity of at least 86%, preferably at least 90% and more preferably at least 94% with SEQ ID NO: 2, where each change in the homologous amino acid sequence is selected from insertion, addition, deletion and substitution of one or more amino acids. Homologs with SEQ ID NO:2 are selected from the enzyme class identified by number EC 1.10.3.1 in the KEGG database (catechol oxidase: diphenol oxidase: o-diphenolase: polyphenol oxidase: pyrocatechol oxidase: dopa oxidase: catecholase: o-diphenol:oxygen oxidoreductase: o-diphenol oxidoreductase). The homologous amino acid sequence is preferably SEQ ID NO: 3, which is also referred to hereinafter as RscK60-del oxidase. In other words, preference is given to a process for producing HTS, which is characterized in that the amino acid sequence homologous with SEQ ID NO: 2 is SEQ ID NO: 3. The homology of SEQ ID NO: 3 with SEQ ID NO: 2 is 91.3%, since the sequence of RscK60 oxidase is 47 amino acids longer.

Sequence identity is defined as the percentage of a homologous amino acid sequence identical to amino acid positions 1 to 543 of the amino acid sequence annotated as polyphenol oxidase/catechol oxidase in SEQ ID NO: 2, where each change in the homologous amino acid sequence is selected from insertion, addition, deletion and substitution of one or more amino acids.

The invention encompasses all engineered variants of the DNA sequence of SEQ ID NO: 1 which are conceivable on the basis of what is called the degenerate genetic code, which encode a protein having an amino acid sequence corresponding to SEQ ID NO: 2 or to a variant homologous with SEQ ID NO: 2, and which have oxidase activity.

The process of the invention is further characterized in that it comprises erythorbic acid, or one of the inexpensive salts thereof, such as the sodium salts sodium D-erythorbate, or the monohydrate thereof, sodium D-erythorbate x H₂O, in order to maximize the yield of HTS as protective substance. As shown in table 3 of example 3, tyrosol was fully converted within a short time in reaction mixtures without sodium D-erythorbate, but the HTS yield was low at 40%. HTS was thus apparently degraded in the reaction mixture. In a parallel reaction mixture in the presence of sodium D-erythorbate x H₂O, by contrast, the tyrosol used was fully transformed to HTS. Sodium D-erythorbate as protective substance thus prevented the degradation of HTS.

The results showed firstly that RscK60-del oxidase could be produced efficiently as recombinant enzyme in E. coli and was directly suitable in the form of resuspended cells, without digestion of the cells, for the biotransformation of tyrosol to HTS, which is one crucial factor for the economic viability of the process. A further unexpected result of the experiment, and one that has not been described to date, was that the addition of sodium D-ery thorbate x H₂O constituted an effective measure for maximization of the yield in the biotransformation of tyrosol to HTS by RscK60-del oxidase.

The amount of erythorbic acid or erythorbate used in the process of the invention is dependent firstly on the dosage of the tyrosol reactant in the reaction mixture and secondly on the tolerance of the enzyme for erythorbic acid or erythorbate.

The prior art, for example EP 3 234 164 B1 and CN 101624607 B, discloses processes for producing HTS in which ascorbic acid is used as protective substance in order to maximize the yield of HTS. But ascorbic acid is also known in the prior art as an inhibitor of tyrosinases, which limits the use amount both of the protective substance and of the tyrosol reactant and hence limits the economic viability of the process. The maximum use amount of ascorbic acid was at a concentration of 0.4 M. Furthermore, in EP 3 234 164 B1, the molar ratio of tyrosol to ascorbic acid was 1:2. In CN 101624607 B, the molar ratio of tyrosol to ascorbic acid was 1:1.57. This means that both processes entail a comparatively high use of ascorbic acid in relation to the tyrosol reactant, which has an adverse effect on the economic viability of the processes.

It has now been found that, surprisingly, as disclosed in examples 4 to 7, in the process of the invention, erythorbate can be used in concentrations exceeding 0.8 M, i.e. in far higher concentrations than known for ascorbic acid or its salt, without inhibiting the activity of the oxidase. Compared to the prior art, this enables the conversion of far higher concentrations of tyrosol to HTS, which is a crucial factor for the economic viability of the process.

For that reason, the process for producing HTS is preferably characterized in that the reaction mixture contains erythorbic acid or erythorbate in a concentration of at least 0.4 M, more preferably of at least 0.6 M and especially preferably of at least 0.8 M.

As shown in table 6, it was possible to convert the tyrosol used virtually completely to HTS with a molar yield of 96.5%. Compared to the prior art such as EP 2 774 909 B1, the process of the invention with RscK60 oxidase or a corresponding homolog and erythorbic acid or ery thorbate as protective substance constitutes an unexpected improvement, since the use of the protective substance in a molar ratio of 1:1 relative to tyrosol was sufficient for the full conversion of >200 mM tyrosol, whereas, according to the prior art, a 2-fold molar excess of sodium ascorbate was required to convert 150 mM tyrosol. Erythorbate or erythorbic acid in combination with RscK60 oxidase or the corresponding homolog is therefore of much better suitability than ascorbate and an engineered tyrosinase variant in order to produce HTS in a biotransformation with high yields of tyrosol.

As shown in table 7, the biotransformation afforded 433 mM (66.7 g/L) HTS from the amount of 434 mM tyrosol (60 g/L) used, which corresponded to a molar yield of 99.7%. Table 8 shows that 707 mM (109 g/L) HTS was obtained from the amount of 724 mM (100 g/L) tyrosol used, which corresponded to a molar yield of 97.6%. This constitutes a distinct improvement over prior art documents such as EP 3 234 164 B1 and CN 101624607 B, in which only 175 mM and 180 mM tyrosol respectively were converted to HTS.

The aim of the biotransformation is a maximum conversion of the tyrosol reactant to the HTS product, i.e. a maximum yield of HTS relative to the amount of tyrosol used. Preference is given to an HTS yield of the biotransformation of >80%, preferably >90%, more preferably >95% and especially preferably 100%, based on the molar amount of tyrosol used. The yield is determined by quantitative HPLC of tyrosol and HTS, as described in example 2.

The process of the invention is further characterized in that the reaction mixture must be supplied with oxygen, in the form of atmospheric oxygen, compressed air or pure oxygen. Oxygen can be introduced here by passive introduction, for example by shaking on an incubation shaker (laboratory scale) or stirring. Oxygen can also be introduced by active introduction of compressed air or oxygen via a sparging tube, or else by a combination of passive and active introduction. Preference is given to the introduction of oxygen by a combination of passive and active introduction.

In addition, the process according to the invention is conducted under defined conditions of pH and temperature. Preference is given to a pH range of the reaction mixture of 5.0 to 8.5; more preferably of 5.5 to 8.0 and more preferably of 6.0 to 7.5. The preferred temperature range is 20° C. to 60° C., more preferably 25° C. to 50° C. and especially preferably from 30° C. to 40° C.

The reaction time until complete conversion of the tyrosol reactant to the HTS product depends on the amount of the reactant used and on the amount of the enzyme used, and is not more than 6 h, preferably not more than 25 h, more preferably not more than 50 h and especially preferably not more than 80 h.

The scale of the preparative reaction mixture is at least 0.5 L, preferably at least 50 L, more preferably 500 L and especially preferably at least 5000 L.

There are various tests available for molar determination of the oxidase activity of a protein. For example, it is possible to use the L-DOPA test disclosed in Behbahani et al. (1993), Microchemical J. 47: 251-260, in which the oxidation of the L-DOPA substrate (3,4-dihydroxy-L-phenylalanine, CAS number 59-92-7) to the dopachrome chromophore (CAS number 3571-34-4) is monitored at a wavelength of 475 nm. As described in example 2 (L-DOPA test) of the present invention, for this purpose, a volume of enzyme solution (cell suspension, isolated cells, cell homogenate or cell-free enzyme extract) containing 4 mg of protein is admixed with a volume of KPi buffer (50 mM potassium phosphate, 1 mM EDTA, pH 6.5) containing 10 mM L-DOPA. The test batches are incubated at 37° ° C. and 140 rpm. After 0, 30, 60 and 120 min, aliquots of the test batches are taken, the solid constituents are separated off, for example by centrifugation, and the absorbance of the supernatant is determined by spectrophotometry at 475 nm.

As described in example 2, oxidase activity can also be detected and quantified in the HPLC test. The test batch, for every 10 mL of batch volume, contains 0.4 mg/ml of protein (or the corresponding amount of cell suspension, isolated cells, cell homogenate or cell-free enzyme extract), 5.1 mM tyrosol and 0 or 10 mM sodium D-erythorbate in KPiE buffer (50) mM potassium phosphate. 10 mM EDTA, pH 6.5). The test batches are incubated at 30° C. and 140 rpm. After 0), 1, 2 and 4 h, aliquots of the test batches are taken and, in order to stop the reaction, 10% (v/v) conc. H₃PO₄ is added immediately in each case. After the solid constituents have been separated of, for example by centrifugation, the supernatant is used for the determination of tyrosol and HTS by means of a correspondingly calibrated HPLC (as known to the person skilled in the art or described in more detail in example 2).

The enzyme activity can be determined directly in the culture broth without reisolating the cells (cell suspension) or after reisolating the cells (isolated cells). In addition, the enzyme activity can be determined in a cell homogenate after digestion of the cells, in which case the cell homogenate can be produced directly from the culture broth or after reisolation of the cells. Furthermore, the determination of the enzyme activity is also possible in a cell extract by removal of particulate cell constituents from the homogenate, for example by centrifugation. Finally, the enzyme can be isolated from the cell extract in a manner known per se, for example by column chromatography, and used as purified protein for determination of the enzyme activity.

The enzyme activity is preferably determined directly from the culture broth, from the cells after reisolation or from a cell homogenate, more preferably from the culture broth or from the cells after reisolation, and especially preferably directly from the culture broth.

In the genetic information for the strain Ralstonia solanacearum K60 published by Remenant et al. (2012), J. Bact. 194: 2742-2743 (GenBank CAGT01000120.1), the cds was identified by SEQ ID NO: 1, which is accessible via the GenBank Locus Tag RSK60_20060005, nt 3460-5091, encoding a protein with NCBI protein database accession number CCF97399.1 (SEQ ID NO: 2). The function assigned to the protein therein was that of a “putative polyphenol oxidase, catechol oxidase”. However, the enzymatic function of the protein arising from the annotation had not been examined experimentally to date and hence has not been characterized to date. On the basis of the annotation as polyphenol oxidase/catechol oxidase, the cds with the DNA sequence from SEQ ID NO: 1 was identified as rscK60-cds, and the protein with the protein sequence from SEQ ID NO: 2 in the present invention as RscK60 oxidase.

It has been found that, surprisingly, both the protein encoded by SEQ ID NO: 1 with SEQ ID NO: 2 (RscK60 oxidase) and an amino acid sequence homologous with SEQ ID NO 2 have the oxidase activity of the invention, i.e. can convert tyrosol to HTS in the presence of atmospheric oxygen without being inhibited by erythorbic acid or erythorbate in concentrations of >0.4 M.

Since the tyrosinase from the R. solanacearum strain GMI1000 disclosed in EP 3 234 164 B1 (Salanoubat et al. 2002, Nature 415: 497-502, Hernandez-Romero et al. 2006, FEBS J. 273: 257-270, Molloy et al. 2013, Biotechnol. and Bioengineering 110: 1849-1857), or an engineered variant of that enzyme, can also be used for the biotransformation of tyrosol to HTS, a sequence comparison was conducted. On comparison of the amino acid sequence of the RscK60 oxidase annotated as polyphenol oxidase/catechol oxidase (SEQ ID NO: 2) with the tyrosinase from R. solanacearum GMI1000 (Genbank accession number NP_518458) from EP 3 234 164 B1, distinct differences were found. Homology is only about 85%, since the sequence of the RscK60 oxidase annotated as polyphenol oxidase/catechol oxidase from R. solanacearum K60 is 47 amino acids longer than that of the tyrosinase from R. solanacearum GMI1000 and additionally differs in 34 further amino acids.

A protein with the amino acid sequence of SEQ ID NO: 2 or an amino acid sequence homologous to SEQ ID NO: 2 is particularly suitable for the production of HTS, especially on an industrial scale, because:
the oxidase, for production purposes, can be produced by fermentation on a scale of 1 L or more.
tyrosol in a concentration of >0.2 M is converted to HTS.
the protective substance, i.e. erythorbic acid or erythorbate, by contrast with ascorbic acid or its ascorbate salt, can be used in concentrations of >0.4 M for avoidance of the degradation of HTS.
the molar ratio of the tyrosol reactant to erythorbic acid or erythorbate in the mixture is only 1:1 to 1:1.2, which has a cost-saving effect on the process by comparison with the prior art, where the molar ratio of tyrosol to ascorbic acid or ascorbate is 1:1.57 to 1:2.
the oxidase can be used in the form of fermenter broth without further workup, which dispenses with the costly reisolation and workup of the fermenter cells.

In summary, the providing of the oxidase of the invention enables more economically viable biotechnological production of HTS.

The protein (iii) with oxidase activity and the amino acid sequence of SEQ ID NO: 2 or an amino acid sequence homologous to SEQ ID NO: 2 can be produced by fermentation or by chemical synthesis of the amino acid sequence.

The process for producing HTS by enzymatic conversion of tyrosol to HTS is preferably characterized in that the oxidase (iii) is produced by recombinant means by fermentation in E. coli. This gives rise to a fermenter broth.

Fermentation is a process step for production of cell cultures on a laboratory scale or on an industrial scale, by inducing a microbial production strain containing the gene construct for expression of the oxidase enzyme to grow under defined conditions of culture medium, temperature, pH, oxygen supply and medium mixing, in order to achieve a maximum cell density and maximal activity of the protein/enzyme to be produced in the cell culture. The terms “laboratory scale” or “industrial scale” differ merely by the size of culture. For instance, a batch volume of less than 1000 mL is referred to as the laboratory scale (described, for example, as a shaken flask culture), whereas a batch volume over and above 1000 mL is referred to as the industrial scale.

For the fermentation, first of all, a gene construct is produced by cloning the cds of RscK60 oxidase (SEQ ID NO: 1), that of the engineered variant RscK60-del (nt 142-1632 of SEQ ID NO: 1), the cds encoding a protein homologous with SEQ ID NO: 2, or the cds of a protein to be tested into an expression vector, meaning that the gene construct contains all the information to express a protein from the cloned cds.

The person skilled in the art is aware of various ways of producing suitable gene constructs. Preference is given to the expression vector pKKj, disclosed in EP 2 670 837 A1. pKKj contains the known tac promoter, such that the expression of coding gene sequences functionally linked to that tac promoter can be induced by addition of the IPTG inductor (isopropyl-β-thiogalactoside). Gene constructs of the invention are pRscK60 (FIG. 1) and pRscK60-del (FIG. 2).

Then a production strain is produced for the corresponding oxidase by transforming the corresponding gene construct in a known manner into a microorganism (production host) suitable for protein production. The production host is preferably selected from the Escherichia coli species, and is especially preferably a microorganism from the E. coli K12 JM105 strain (commercially available under strain number DSM 3949 from the DSMZ German Collection of Microorganisms and Cell Cultures GmbH). The production strain is more preferably E. coli JM105 x pRscK60-del.

RscK60 oxidase, or an oxidase homologous with SEQ ID NO: 2, is expressed by culturing the production strain in a culture medium. The culture may be on a laboratory scale by means of a shaken flask culture (as known to the person skilled in the art and described in example 3) or on an industrial scale by fermentation (as known to the person skilled in the art and described in example 4), and the oxidase activity of an aliquot of the resulting culture broth may be examined.

In the fermentative method of producing an oxidase of the invention of SEQ ID NO: 2 or a sequence homologous with SEQ ID NO: 2, firstly biomass of the production strain and secondly the oxidase are formed. Formation of biomass and oxidase may correlate here in time or else be decoupled from one another in time, in that the enzyme production, after formation of the biomass in a first fermentation phase, is started in the second phase by an inductor of gene expression. Preference is given to a fermentation method as disclosed in example 4, in which the formation of biomass and oxidase are decoupled from one another in time and the production of the oxidase is started by an inductor. A preferred inductor is IPTG (isopropyl-β-thiogalactoside).

The process for producing HTS by enzymatic conversion of tyrosol to HTS is preferably characterized in that the oxidase (iii) is produced by fermentation on an industrial scale, more preferably by fermentation with a fermentation volume of greater than 1 L, especially preferably greater than 10 L, specifically preferably greater than 1000 L and further preferably greater than 5000 L.

The terms “culture”, “growth” and “fermentation”, and also, for example, “culture medium”, “growth medium” and “fermentation medium”, are used synonymously in the context of the present invention. The term “culture broth” or “fermenter broth” refers to the end product of the fermentation containing the cells that contain oxidase (iii) and the cell culture medium.

Culture media are familiar to the person skilled in the art from practical microbial culturing. They typically consist of a carbon source (C source), a nitrogen source (N source), and additions such as vitamins, salts and trace elements that optimize cell growth and the production of the oxidase.

C sources are those that can be utilized by the production strain for formation of biomass. A preferred C source is glucose.

N sources are those that can be utilized by the production strain for biomass formation. Preferred N sources are ammonia, in gaseous form or in aqueous solution as NH₄OH, or else salts thereof, for example ammonium sulfate or ammonium chloride. The N sources also include complex amino acid mixtures, preferably including yeast extract, proteose peptone or corn steep liquor, the latter in liquid form or else in a dried form called CSD.

The culturing can be effected in what is called batch mode, wherein the culture medium is inoculated with a starter culture of the production strain and then cell growth proceeds without further feeding of nutrient sources.

The culturing can also be effected in what is called fed batch mode, as disclosed in example 4, wherein, after an initial phase of growth in batch mode, nutrient sources are additionally fed in, in order to compensate for the consumption thereof. The feed may consist of the C source, the N source, one or more vitamins that are important for production, or trace elements, including preferably Cu(II) ions, or of a combination of the above. The feed components may be metered in together as a mixture or else separately in individual feeds. In addition, the inductor may also be added to the feed. The feed may be fed in continuously or in portions (discontinuously), or else in a combination of continuous and discontinuous feeding. Preference is given to culturing by the fed batch mode.

A preferred C source in the feed is glucose.

The C source is preferably metered into the culture such that the content of the carbon source in the fermenter during the production phase does not exceed 10 g/L. Preference is given to a maximum concentration of 2 g/L, more preferably of 0.5 g/L, especially preferably of 0.1 g/L.

Preferred N sources in the feed are ammonia, in gaseous form or in aqueous solution as NH₄OH.

Further media additions added may be salts of the elements phosphorus, chlorine, sodium, magnesium, nitrogen, potassium, calcium, iron, and traces (i.e. in μM concentrations) of salts of the elements molybdenum, boron, cobalt, manganese, zinc, copper and nickel. In addition, it is possible to add organic acids (e.g. acetate, citrate), amino acids (e.g. isoleucine) and vitamins (e.g. vitamin B1, vitamin B6) to the medium.

The culturing is effected under pH and temperature conditions that promote the growth of the production strain and gene expression. The useful pH range is from pH 5 to pH 9. Preference is given to a pH range from pH 5.5 to pH 8. Particular preference is given to a pH range from pH 6.0 to pH 7.5.

The preferred temperature range for the growth of the production strain is 20° C. to 40° C. Particular preference is given to the temperature range from 25° C. to 37° C. and especially preferably from 28° C. to 34° C.

The production strain can optionally be grown without supply of oxygen (anaerobic culturing) or else with supply of oxygen (aerobic culturing). Preference is given to aerobic culturing with oxygen.

In the case of aerobic culturing, saturation of the oxygen content of at least 10% (v/v), preferably of at least 20% (v/v) and more preferably of at least 30% (v/v) is established. Oxygen saturation in the culture is regulated in accordance with the prior art automatically via a combination of gas supply and stirring speed.

Oxygen supply is assured by introduction of compressed air or pure oxygen. Preference is given to aerobic culturing by introduction of compressed air. The useful range for compressed air supply in aerobic culturing is 0.05 vvm to 10 vvm (vvm: input of compressed air into the fermentation batch reported in liters of compressed air per liter of fermentation volume per minute). Preference is given to introduction of compressed air at 0.2 vvm to 8 vvm, more preferably at 0.4 to 6 vvm and especially preferably at 0.8 to 5 vvm.

The maximum stirring speed is 2500 rpm, preferably 2000 rpm and more preferably 1800 rpm. Protein production is induced by addition of IPTG. Preference is given to the addition of IPTG in a concentration of at least 0.1 mM, more preferably at least 0.2 mM and especially preferably at least 0.4 mM. The inductor can be added in one portion, divided into multiple portions, or else continuously. Preference is given to the addition of the IPTG inductor in one portion.

IPTG can be added directly at the start of the fermentation or once the cell density in the fermenter has reached a particular threshold value. The cell density, referred to as OD₆₀₀, is determined here in a known manner by photometry, by measuring the absorbance/mL of fermenter broth at 600 nm (OD₆₀₀: optical density at 600 nm/mL of fermenter broth). Preference is given to the addition of IPTG from an OD₆₀₀ of 10/mL, more preferably from an OD₆₀₀ of 30/mL and especially preferably from an OD₆₀₀ of 50/mL.

The culture time is between 10 h and 100 h. Preference is given to a culture time of 20 h to 70 h. Particular preference is given to a culture time of 25 h to 50 h.

Culture batches that are obtained by the method described above contain the RscK60 oxidase or the corresponding homolog. The oxidase can be used further directly as fermenter broth in the process of the invention without further workup, as a cell suspension after reisolation of the cells, as a cell homogenate after digestion of the cells, either directly from the fermenter broth or after reisolation of the cells, as cell-free enzyme extract, or else as an enzyme purified therefrom. Preference is given to the direct use of the oxidase in the process of the invention as fermenter broth without further workup, after reisolation of the cells as cell suspension, or as cell homogenate after digestion of the cells. Particular preference is given to the direct use of the oxidase in the process of the invention as fermenter broth without further workup or after reisolation of the cells as cell suspension. Especially preferred is the direct use of the oxidase in the process of the invention as fermenter broth without further workup.

The culturing may be on a laboratory scale by shaken flask culturing (as described in example 3) or on an industrial scale by fermentation (as described in example 4), with the aim of producing a cell culture with maximum enzyme activity based on the transformation of tyrosol to HTS. A maximum enzyme activity is achieved firstly by means of a growth medium that promotes good cell growth, and by means of further additions that selectively stimulate enzyme production. A known method of stimulating enzyme production is based on gene constructs with inducible promoters, as present in the expression vector pKKj used in example 1. The use of the expression vector pKKj is disclosed, for example, in EP 2 670 837 A1. pKKj features the known tac promoter. The expression of genes functionally linked to the tac promoter can be greatly enhanced by addition of the IPTG inductor (isopropyl-β-thiogalactoside).

A further means of increasing enzyme activity is the addition of cofactors of the enzyme activity. Since only the gene sequence and no experimental studies of enzyme activity were known for the RscK60 gene of the invention, various means of enhancing enzyme activity were examined. The annotation of the RscK60 gene as polyphenol oxidase/catechol oxidase suggested metal dependence of the enzyme activity. For example, the effect of the addition of Cu(II) ions in the culture medium on enzyme production was examined. It was found that, surprisingly, an elevated concentration of Cu(II) ions led to a significant increase in enzyme activity (example 3). The RscK60 oxidase differs here from the prior art, where, for example, for production of the enzyme in EP 3 234 164 B1, Cu(II) ions were not used to increase the enzyme yield, even though the dependence on copper thereof was known from the technical literature (Hernandez-Romero et al. (2006), FEBS J. 273: 257-270, Molloy et al. (2013), Biotechnol. and Bioengineering 110: 1849-1857).

The open reading frame (ORF, synonymous with cds, coding sequence) refers to that region of the DNA or RNA that begins with a start codon and ends with a stop codon and encodes the amino acid sequence of a protein. The ORF is also referred to as coding region, where the stop codon is not translated into an amino acid.

Cds are surrounded by non-coding regions. The gene refers to the section of DNA that contains all the basic information for production of a biologically active RNA. A gene contains the DNA section from which a single-strand RNA copy is produced by transcription, and the expression signals involved in the regulation of this copying operation. The expression signals include, for example, at least one promoter, a transcription start, a translation start and a ribosomal binding site. Further expression signals that are possible are a terminator and one or more operators.

A gene construct refers in the context of the invention to a circular DNA molecule (plasmid, expression vector) in which the cds of a gene is linked to further genetic elements (e.g. promoter, terminator, selection marker, replication origin). The genetic elements of the gene construct firstly result in extrachromosomal inheritance thereof during cell growth, and the production of the protein encoded by the gene.

The abbreviation WT (Wt) denotes the wild type. The wild-type gene refers to the form of the gene that has evolved naturally and is present in the wild-type genome. The DNA sequence of Wt genes is publicly available in databases such as NCBI.

An engineered variant/functional derivative/genetically produced variant of the enzyme defines enzyme variants that arise through mutation, i.e. as a result of changes in the nucleotide sequence from the DNA of the Wt gene and lead to an enzyme having modified protein sequence, where the modified protein sequence may comprise any desired change from insertion, addition, deletion and substitution of amino acids, provided that the original enzyme function is conserved.

The process for producing HTS in the context of the invention is preferably a biotransformation process and is more preferably composed of the following operating steps: in a first step an oxidase enzyme in recombinant form is produced by fermentation, in a second step the resulting fermenter broth is reacted directly without further workup in a reaction mixture together with the tyrosol reactant (starting material) and further auxiliaries, and in a third step the HTS product is isolated from the reaction mixture without additional working steps by extraction with a solvent, followed by the distillative removal of the solvent.

Biotransformation is defined as transformation of a reactant to a product under enzymatic catalysis.

Extraction is defined as a process step in which the reaction mixture is mixed with a liquid (extractant) that is insoluble therein and hence the product of the reaction is transferred into the extractant. After separation of reaction mixture and extractant (phase separation), the product can be isolated by removing the extractant.

Annotation in genetics and bioinformatics refers to a functional assignment that can originate either from experimental findings or from a computer-assisted prediction. The annotation of a DNA sequence describes, inter alia, the protein-encoding regions (cds) including the encoded proteins in that sequence.

In a preferred embodiment, the process for producing HTS by enzymatic conversion of tyrosol to HTS is characterized in that the fermentation for production of the oxidase is effected in the presence of a concentration of at least 0.02 mM, more preferably at least 0.1 mM, especially preferably at least 0.2 mM and specifically preferably at least 0.5 mM Cu(II) ions. The Cu(II) ions may be provided here by any known Cu(II) salt, for example by Cu(II) sulfate, Cu(II) chloride, Cu(II) acetate or Cu(II) nitrate, preference being given to Cu(II) sulfate, either in anhydrous form or in pentahydrate form (CuSO₄ x 5 H₂O).

An increase in the content of Cu(II) ions in the culture medium has the advantage that an elevated yield of enzymatic activity can be achieved.

As summarized in table 2 (example 3), the oxidase enzyme activity was increased by more than tenfold by supplementing the culture medium with Cu(II) ions in the form of CuSO₄ x 5 H₂O. Supplementing the culture medium with Cu(II) ions thus constitutes an efficient method that has not yet been described in the prior art in order to optimize the production of an enzyme suitable for the production of HTS.

Moreover, it is preferable that the fermenter broth from the fermentation for production of the oxidase is used directly in the process for producing HTS without further workup.

As shown by table 4 of example 4, the RscK60-del oxidase can be produced for industrial use by recombinant means in E. coli and used directly as fermenter broth without further isolation or digestion of the cells in the biotransformation of tyrosol to HTS. In the case of a sufficiently high dosage of the fermenter cells, tyrosol was converted quantitatively in a concentration of 180 mM, which is very high compared to the prior art, with HTS as product (table 5). Unexpectedly by comparison with the prior art with ascorbic acid as protective substance, a dosage of the sodium D-ery thorbate x H₂O protective substance in a molar ratio of 1:1 was sufficient to suppress the degradation of HTS.

For the purposes of maximum economic viability, tyrosol is to be converted in a maximum concentration in the biotransformation to give HTS. In a preferred embodiment, the process for producing HTS is characterized in that tyrosol is used in a concentration of more than 200 mM, more preferably more than 400 mM and especially preferably more than 700 mM.

It is preferable that, in the process for preparing HTS, the molar ratio of tyrosol to the amount of erythorbic acid or erythorbate used is not more than 1:1.50, more preferably not more than 1:1.2, especially preferably not more than 1:1 and specifically preferably not more than 1:0.5.

Furthermore, it is preferable that, in the process for producing HTS, the proportion by volume of the fermenter broth from the fermentation to produce the oxidase in the reaction mixture is up to 90%, more preferably not more than 50%.

The process for producing HTS is preferably characterized in that the reaction mixture is reacted for a period of time until at least 90% of the tyrosol used is converted to HTS. The time specifically required is dependent on the tyrosol dosage. It is determined by determining the amount of tyrosol and HTS by HPLC (for description see example 2, HPLC test).

For example, 70 g/L tyrosol was converted to an extent of 95% within 24 h. The tyrosol used is preferably converted to HTS to an extent of at least 80% within 24 h.

In the process of the invention for producing HTS, it is preferable that HTS is produced from tyrosol with a molar yield of preferably at least 70%, more preferably at least 90% and especially preferably at least 95%.

For isolation of HTS from the reaction mixture, the reaction mixture is mixed with an immiscible solvent without intermediate step and, after phase separation, the product-containing solvent phase is separated off. Solvents suitable for extraction of HTS are known from EP 2 774 909 B1. A solvent preferred for extraction is ethyl acetate (CAS number 141-78-6). The extraction, preferably with ethyl acetate, can be repeated as often as desired until complete removal of the HTS from the reaction mixture. Alternatively, the extraction, preferably with ethyl acetate, can also be conducted continuously in a known manner, for example by countercurrent extraction. For better phase separation, the mixture can be pretreated before the extraction, for example by acidifying with sulfuric acid, by heating, or a combination of the two measures, in order to denature the proteins present in the mixture.

The process for producing HTS is preferably characterized in that HTS is isolated from the reaction mixture by extraction with ethyl acetate. More preferably, the ethyl acetate is then separated off by distillation.

The extraction is preferably effected at neutral pH, i.e. at a pH 6.5-7.5.

After distillative removal of the solvent (ethyl acetate), HTS is obtained in high yield and purity. The molar yield, based on the amount of tyrosol used, is preferably >60%, more preferably >70% and especially preferably >80%. The process for producing HTS is preferably characterized in that HTS is isolated from the reaction mixture with a purity of at least 80%, more preferably at least 90% and especially preferably at least 93%.

Various analytical methods for identification, quantification and determination of the purity of the tyrosol reactant and of the HTS product are available, including spectrophotometry, NMR, gas chromatography, HPLC, mass spectroscopy, gravimetry, elemental analysis, or else a combination of these analysis methods.

In summary, and as demonstrated by the examples, the process of the invention for producing HTS with the new process components RscK60-del oxidase or a homologous oxidase and erythorbic acid or erythorbate has an unexpected improvement over the prior art such as EP 2 774 909 B1 and CN 101624607 B. Even a small amount of the erythorbic acid or erythorbate protective substance used is sufficient for the full conversion of significant amounts of tyrosol (examples show: tyrosol in a molar ratio to erythorbate of 1:1 to 1:1.2 is sufficient for the complete conversion of >400 mM tyrosol), whereas, according to the prior art, a 2-fold and 1.57-fold molar excess of sodium ascorbate, or ascorbic acid, over tyrosol was required to convert 150 mM and 180 mM tyrosol respectively. As a result, the process of the invention is efficient and inexpensive. It enables the production of HTS with unexpectedly high volume yields and high purity.

The process of the invention may additionally comprise further inexpensive and simple process steps, namely the use of the cell culture broth without further workup from the fermentation of a strain that produces RscK60-del oxidase or a corresponding homolog of said oxidase in the biotransformation, and product isolation by extraction directly from the reaction mixture.

### BRIEF ELUCIDATION OF THE FIGURES

FIG. 1 shows the 4.5 kb vector pRscK60 produced in example 1.

FIG. 2 shows the 4.4 kb vector pRscK60-del produced in example 1.

### ABBREVIATIONS USED IN THE APPLICATON

cds coding DNA sequence (see above for definition)
nt nucleotide(s)
HTS hydroxytyrosol
HPLC high-performance liquid chromatogrphy

The invention is elucidated by the examples that follow without being restricted thereby:

### EXAMPLE 1

### Production of the Expression Vectors pRscK60 and pRscK60-Del

For the isolation of the cds of the RscK60 gene, genomic DNA from the Ralstonia solanacearum K60 strain (commercially available under strain number DSM 9544 at the DSMZ German Collection of Microorganisms and Cell Cultures GmbH) was used.

The coding sequence of RscK60 to be isolated (referred to hereinafter as rsck60-cds, SEQ ID NO: 1), encoding a putative polyphenol oxidase/catechol oxidase, is disclosed in the NCBI (National Center for Biotechnology Information) nucleotide database under Locus Tag CAGT01000120.1, nt 3460-5091 (SEQ ID NO: 1), encoding a protein with Genbank accession number CCF97399.1 (SEQ ID NO: 2), referred to hereinafter as RscK60 oxidase.

The vectors pRscK60 and pRscK60-del were produced using the following DNA fractions from the putative cds of the polyphenol oxidase/catechol oxidase:
rsck60-cds: SEQ ID NO: 1 nt 1-nt 1632, encoding a protein with amino acid sequence SEQ ID NO: 2, referred to hereinafter as RscK60 oxidase.
rscK60-del-cds: SEQ ID NO: 1 nt 142-nt 1632, encoding a protein with amino acid sequence SEQ ID NO: 3, referred to hereinafter as RscK60-del oxidase. RscK60-del oxidase is a protein with N-terminal truncation by 47 amino acids compared to RscK60 oxidase.

The DNA fragment rscK60-cds was isolated in a PCR reaction (“Phusion™ High-Fidelity” DNA polymerase, Thermo Scientific™) as a 1.6 kb fragment. For this purpose, genomic DNA from the R. solanacearum K60 strain and the primers rsck60-If (SEQ ID NO: 4) and rsck60-2r (SEQ ID NO: 5) were used.

The DNA fragment rscK60-del-cds was isolated in a PCR reaction (“Phusion™ High-Fidelity” DNA polymerase, Thermo Scientific™) as a 1.5 kb fragment. For this purpose, genomic DNA from the R. solanacearum K60 strain and the primers rsck60-3f (SEQ ID NO: 6) and rsck60-2r (SEQ ID NO: 5) were used.

Primer rsck60-1f contains an EcoRI cleavage site adjoined by 23 nucleotides (nt) beginning with the start of the cds of RscK60 oxidase (nt 1-23 in SEQ ID NO: 1).

Primer rsck60-2r contains a HindIII cleavage site adjoined by 24 nucleotides (nt) from the 3′ region of the cds of RscK60 oxidase (nt 1609-1632 in SEQ ID NO: 1, in reversed complementary form).

Primer rsck60-3f contains an EcoRI cleavage site adjoined by 24 nucleotides (nt) from the 5° region of the cds of RscK60 oxidase (nt 142-165 in SEQ ID NO: 1).

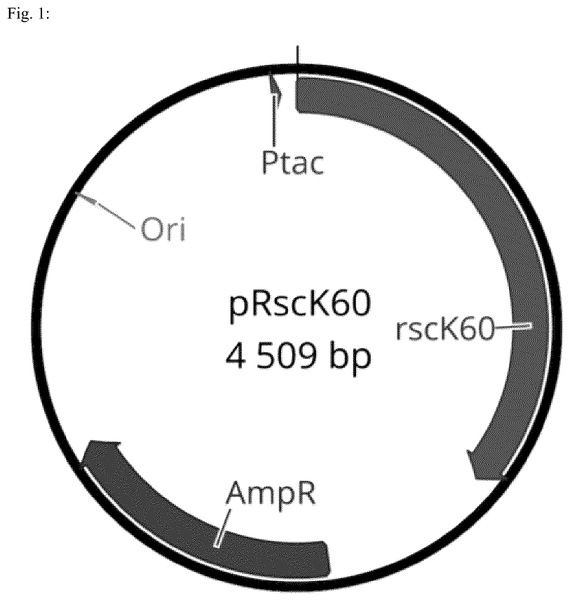

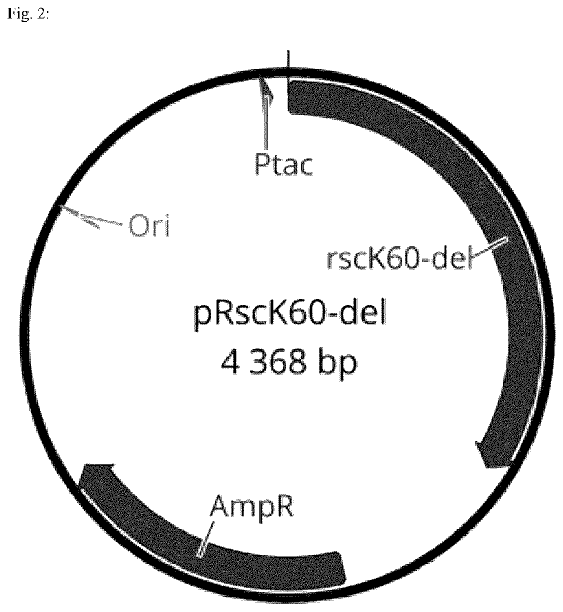

The PCR products were cleaved with EcoRI (present in the primers rsck60-If and rsck60-3f) and HindIII (present in primer rsck60-2r) and cloned into the pKKj expression vector that had been cleaved beforehand with EcoRI and HindIII. This gave rise to the 4.5 kb expression vector pRscK60 (FIG. 1) and the 4.4 kb expression vector pRscK60-del (FIG. 2).

The expression vector pKKj, disclosed in EP 2 670 837 A1, is a derivative of the expression vector pKK223-3. The DNA sequence of pKK223-3 is disclosed in the GenBank gene database under accession number M77749.1. About 1.7 kb were removed from the 4.6 kb plasmid (bp 262-1947 of the DNA sequence disclosed in M77749.1), which gave rise to the 2.9 kb expression vector pKKj.

### EXAMPLE 2

### Analytical Tests

### Cell Suspensions and Isolated Cells:

Cells cultured in a shaken flask (example 3) or in a fermenter (example 4) were either used directly as cell suspensions (culture broth, fermenter broth) without further isolation for analytical tests or the cells were isolated.

The cells were isolated from a suspension (culture broth, fermenter broth) by centrifugation of the suspension (10 min 15 000 rpm, Sorvall RC5C centrifuge, equipped with an SS34 rotor). The resultant cell pellet was washed once with 0.9% (w/v) NaCl. For further use as isolated cells, the cell pellet from a 100 mL culture was suspended in 20 mL of KPi buffer (50 mM potassium phosphate, 1 mM EDTA, pH 6.5).

### Production of a Cell Homogenate:

For production of a cell homogenate, the FastPrep-24™ 5G cell homogenizer from MP Biomedicals was used. 2×1 mL of cell suspension was digested in 1.5 mL tubes containing glass beads (“Lysing Matrix B”) that had been prefabricated by the manufacturer (3×20 sec at a shaken frequency of 6000 rpm with breaks of 30 sec in each case between the intervals).

### Production of an Enzyme Extract:

A cell-free enzyme extract was produced from the cell homogenate by centrifugation (10 min 15 000 rpm, Sorvall RC5C centrifuge, equipped with an SS34 rotor) and isolation of the resulting supernatant.

### Determination of the Protein Content of a Sample:

The protein content of cell suspensions, isolated cells, cell homogenates or enzyme extracts was determined with a Qubit 3.0 fluorometer from Thermo Fisher Scientific using the “Qubit® Protein Assay Kit” according to the manufacturer's instructions.

### Determination of the Oxidase Enzyme Activity (L-DOPA Test):

The oxidase enzyme activity was determined using a photometric test in which the oxidation of the L-DOPA enzyme substrate (3,4-dihydroxy-L-phenylalanine, CAS number 59-92-7) to the dopachrome chromophore (CAS number 3571-34-4) at a wavelength of 475 nm is monitored (Behbahani et al. (1993), Microchemical J. 47: 251-260). The enzyme test was conducted with cell suspensions (e.g. monitoring of the progression of production in the fermenter), isolated cells, cell homogenate or cell-free enzyme extract.

A test batch contained, in a 100 mL Erlenmeyer flask, in a batch volume of 8 mL: 4 mL of KPi buffer, 10 mM L-DOPA (Sigma-Aldrich) and 4 mL of the sample to be tested (cell suspension, isolated cells, cell homogenate or cell-free enzyme extract).

If samples from the shaken flask culture were to be tested, the cells were first concentrated by centrifuging 25 mL of shaken flask mixture for isolation of a cell pellet (10 min 15 000 rpm, Sorvall RC5C centrifuge, equipped with an SS34 rotor), the resultant cell pellet was used as described above for production of isolated cells, cell homogenate or cell-free enzyme extract, and, for further use, the volume of the sample to be tested was adjusted to 4 mL with KPi buffer.

If samples from fermentation were to be tested, 1.6 mL of fermentation batch was used directly as cell suspension or as described above for production of isolated cells, cell homogenate or cell-free enzyme extract, and, for further use, the volume of the sample to be tested was adjusted to 4 mL with KPi buffer.

The reaction was started by adding the respective sample to be tested. The test batches were incubated in a shaker (Infors) at 37° C. and 140 rpm. 1 mL aliquots were taken at the time points 0 min, 30 min, 60 min and 120 min, and centrifuged immediately at 13 000 rpm for 5 min (Heraeus™ Fresco™ 21 centrifuge, Thermo Scientific™), and the absorbance of the supernatant was determined at 475 nm (Genesys™ 10S UV-VIS spectrophotometer, Thermo Scientific™).

1 unit (U) of oxidase activity is defined as the amount of enzyme that produces 1 μmol dopachrome/min from L-DOPA under test conditions (extinction coefficient of dopachrome ε₄₇₅ₙₘ=0.37 x 10⁴ L x Mol⁻¹ x cm⁻¹).

The specific oxidase activity was calculated by basing the oxidase enzyme activity on 1 mg of total protein in the measured sample (cell extract, homogenate or cell suspension) (U/mg of protein).

The (specific) oxidase activity determined by L-DOPA test is referred to hereinafter as (specific) oxidase activity in the L-DOPA test.

### Determination of Tyrosol and HTS by HPLC (HPLC Test):

A test biotransformation of tyrosol to HTS was conducted on an analytical scale. Tyrosol solution: 7 mg of tyrosol (Sigma-Aldrich, final concentration 5.1 mM in the test) was weighed into a 100 mL Erlenmeyer flask, dissolved in 4.9 mL of KPiE buffer (50 mM potassium phosphate, 10 mM EDTA, pH 6.5), and supplemented with 0. 1 mL of 1 M sodium D-erythorbate x H₂O (Sigma-Aldrich, final concentration 10 mM in the test). For comparative purposes, in individual tests, 7 mg of tyrosol was dissolved in 5 mL of KPiE buffer without addition of sodium D-ery thorbate.

Test batches: 50 mL of suspension of the cells cultured in a shaken flask, or 10 mL of the cells cultured in the fermenter, was suspended in 5 mL of KPiE buffer and added to the tyrosol solution at the start of the reaction. The test batches (volume 10 mL) were incubated on a shaker (Infors) at 30° C. and 140 rpm. Samples each of 1 mL were taken at the time points 0 h, 1 h, 2 h and 4 h, and, in order to stop the reaction, admixed immediately with 0.1 mL in each case of conc. H₃PO₄. After centrifugation (5 min at 13 000 rpm, Heraeus™ Fresco™ 21 centrifuge, Thermo Scientific™M), 1 mL in each case of the supernatant was dispensed for the determination of tyrosol and HTS by HPLC.

### HPLC Analysis of Tyrosol and HTS:

For quantitative determination of tyrosol and HTS, an HPLC method respectively calibrated for tyrosol and HTS was used. The reference substances tyrosol and HTS for calibration came from Sigma-Aldrich. An Agilent Infinity II HPLC instrument was used, equipped with a diode array detector. The detector was set to the wavelength of 274 nm. In addition, a Luna C18(2) column from Phenomenex, length 250 mm, internal diameter 4.6 mm, particle size 5 μm, was adjusted to a temperature of 30° C. in the column oven. Eluent A: 5 mL of H₃PO₄ in 1 L of H₂O. Eluent B: acetonitrile. The separation was effected in gradient mode from 5% to 10% eluent B within 5 min, followed by 10% to 16% eluent B within 15 min at a flow rate of 1 mL/min. Retention time of tyrosol: 13.9 min: retention time of HTS: 10 min.

The yield of the reaction in the context of the invention is defined as the amount of tyrosol used (reactant) which is converted under reaction conditions to HTS (product). The yield may be reported as volume yield in the absolute amount of product based on volume (mM or g/L) or as relative yield of the product in percent (also referred to as percentage yield), i.e. the absolute yield is based on the tyrosol used (reactant) (taking account of the molecular weights of 138.2 g/mol for tyrosol (reactant) and 154.2 g/mol for HTS (product)).

### EXAMPLE 3

### Expression of RscK60 and of RscK60-Del Oxidase in E. coli by Shaken Flask Culture

For isolation of plasmid DNA, the expression vectors pRscK60 and pRscK60-del from example I were each transformed into a commercially available E. coli strain NEBR 10-beta (New England Biolabs) which is used for cloning purposes. A cell culture was produced (37° C., 120 rpm, Infors tray shaker) for each clone from the transformation by culturing in LBamp medium (10 g/L tryptone, GIBCOTM, 5 g/L yeast extract from BD Biosciences, 5 g/L NaCl, 100 mg/L ampicillin), and plasmid DNA was isolated from the cells with a plasmid DNA isolation kit in accordance with the manufacturer's instructions (QIAprep® Spin Miniprep Kit, Qiagen).

Plasmid DNA of the expression vectors pRscK60 and pRscK60-del was transformed by known methods into the E. coli K12 strain JM105. The E. coli JM105 strain is commercially available under strain number DSM 3949 from DSMZ German Collection of Microorganisms and Cell Cultures GmbH.

Clones for the transformation were selected on Lbamp plates (10 g/L tryptone, GIBCO™, 5 g/L yeast extract from BD Biosciences, 5 g/L NaCl, 15 g/L agar, 100 mg/mL ampicillin) and respectively identified as JM105 x pRscK60 and JM105 x pRscK60-del. The control used was E. coli JM105, transformed with the pKKj vector (E. coli JM105 x pKKj), from which transformants were produced in the same way.

A pre-culture was produced (culture at 37° C. and 120 rpm overnight, Infors tray shaker) for each clone of the E. coli strains JM105 x pKKj, JM105 x pRscK60 and JM105 x pRscK60-del in 30 mL of Lbamp medium (10 g/L tryptone, 5 g/L yeast extract, 5 g/L NaCl, 100 mg/mL ampicillin).

2 mL of each preculture were used as inoculum for a main culture of 100 mL of SM3 medium (1 L Erlenmeyer flask), supplemented with 15 g/L glucose, 0.5 mM CuSO₄ x 5 H₂O and 100 mg/L ampicillin. The main culture was shaken at 30° C. and 140 rpm until a cell density OD₆₀₀ of 2.0 was attained (OD₆₀₀: photometric determination of cell density by determination of absorbance at 600 nm). Then the IPTG inductor (isopropyl-β-thiogalactoside, final concentration 0.4 mM, Sigma-Aldrich) was added and the mixture was shaken at 30° C. and 140 rpm overnight.

Composition of the SM3 medium: 12 g/L K₂HPO₄, 3 g/L KH₂PO₄, 5 g/L (NH₄)₂SO₄, 0.3 g/L MgSO₄ x 7 H₂O, 0.015 g/L CaCl₂ x 2 H₂O, 0.002 g/L FeSO₄ x 7 H₂O, 1 g/L Na₃ citrate x 2 H₂O, 0.1 g/L NaCl: 5 g/L peptone (Oxoid): 2.5 g/L yeast extract (BD Biosciences): 0.005 g/L vitamin B1 (Sigma-Aldrich): 1 mL/L trace element solution.

Composition of the trace element solution: 0.15 g/L Na₂MoO₄ x 2 H₂O, 2.5 g/L H₃BO₃, 0.7 g/L CoCl₂ x 6 H₂O, 0.25 g/L CuSO₄ x 5 H₂O, 1.6 g/L MnCl₂ x 4 H₂O, 0.3 g/L ZnSO₄ x 7 H₂O.

Subsequently, the cells from the shaken flask culturing were used to verify enzyme activity by the HPLC test and the photometric L-DOPA test.

Comparison of Activity of RscK60 Oxidase with the Activity of RscK60-Del Oxidase:

For HPLC tests, the cells from 50 mL of a shaken flask culture of the E. coli JM105x pRscK60 (cell density OD₆₀₀ of 6.4/mL) and JM105 x pRscK60-del (cell density OD₆₀₀ of 5.9/mL) strains were isolated by centrifugation and in each case suspended in 5 mL of KPiE buffer. 5 mL in each case of the isolated and resuspended cells was used in the HPLC test described in example 2.

Two HPLC tests were conducted. One batch contained, in a batch volume of 10 mL: 5 mL of the isolated and resuspended JM105 x pRscK60-del cells from the above-described shaken flask culture, 7 mg of tyrosol (final concentration in the batch 5.1 mM), 4.9 mL KPiE buffer and 0.1 mL of 1 M sodium D-erythorbate x H₂O, dissolved in KPiE buffer (see HPLC test in example 2). The second batch contained, likewise in a batch volume of 10 mL: 5 mL of the isolated and resuspended JM105 x pRscK60 cells from the above-described shaken flask culture, 7 mg of tyrosol, 4.9 mL of KPiE buffer and 0.1 mL of 1 M sodium D-erythorbate x H₂O, dissolved in KPiE buffer. The batches were incubated on a shaker (Infors) at 30° C. and 140 rpm. Samples from the test were taken after 0 h, 2 h, 4 h and 6 h, and analyzed by HPLC. The results are reported in table 1.

TABLE 1
Activity of isolated E. coli K12 JM105 × pRscK60-del
cells that express RscK60-del oxidase and of isolated
E. coli K12 JM105 × pRscK60 cells that express
RscK60 oxidase from the shaken flask culture with regard
to the biotransformation of tyrosol to HTS

| | RscK60-del oxidase | | RscK60 oxidase | |
|---|---|---|---|---|
| Time [h] | Tyrosol [mM] | HTS [mM] | Tyrosol [mM] | HTS [mM] |
| 0 | 5.1 | 0.1 | 4.1 | 0.1 |
| 2 | 2.1 | 3.7 | 2.0 | 2.3 |
| 4 | 0.0 | 5.1 | 1.0 | 3.5 |
| 6 | 0.0 | 5.1 | 0.4 | 4.1 |

### Photometric L-DOPA Test:

The enzyme activity of the JM105 x pRscK60-del strain was determined by comparison with the comparative strain JM105 x pKKj by the photometric L-DOPA test. For cells of the two strains, a cell homogenate was produced (digestion of the cells as described in example 2) and used in the L-DOPA test with L-DOPA as enzyme substrate. The specific enzyme activity, determined in the L-DOPA test with L-DOPA as enzyme substrate, was 1.5 mU/mg for the cell homogenate of the E. coli JM105 x pRscK60-del strain and 0 mU/mg for that of the JM105 x pKKj control strain.

### Variation of the Cu(II) Concentration in the Culture Medium:

In preliminary experiments for optimization of the RscK60-del enzyme activity in the shaken flask culture, one parameter varied was the concentration of the Cu(II) concentration in the culture medium. The supplementation of the SM3 culture medium with Cu(II) ions was found to be advantageous. The SM3 culture medium, from the addition of the trace element solution with 0.25 mg/L CuSO₄ x 5 H₂O (final concentration in the culture medium 1 μM), intrinsically contained only a low content of Cu(II) ions.

The effect of Cu(II) ions on the enzyme activity of RscK60-del oxidase was tested, as described above, by shaken flask culturing of the E. coli JM105 x pRscK60-del strain in SM3 medium, 15 g/L glucose, 100 mg/L ampicillin, to which CuSO₄ x 5 H₂O had additionally been added in concentrations of 0 mM to 0.5 mM. The cells from the batches were used as cell homogenate for the determination of activity by L-DOPA test (table 2).

TABLE 2
Dependence of the oxidase activity determined in the L-DOPA
test on the Cu(II) ion concentration in the culture medium

| CuSO₄ × 5 H₂O | Oxidase activity in the L-DOPA test |
|---|---|
| [mM]* | [mU/mL] |
| 0 | 0.432 |
| 0.02 | 0.649 |
| 0.05 | 0.892 |
| 0.1 | 1.198 |
| 0.2 | 2.631 |
| 0.5 | 5.802 |

*All values are based on the concentration of the Cu(II) ions that have been added to the SM3 medium. The concentration of Cu(II) ions present in the SM3 medium of 1 μm was not taken into account.

### Effect of Sodium D-Erythorbate on HTS Stability:

For HPLC tests, the combined cells from 2 x 50 mL shaken flask cultures of the E. coli JM105 x pRscK60-del strain (cell density OD₆₀₀ of 4.7/mL) were isolated by centrifugation and resuspended in 10 mL of KPiE buffer. 5 mL in each case of the isolated and resuspended cells were used in the HPLC test described in example 2.

Two comparative test biotransformations were conducted. One batch contained, in a batch volume of 10 mL: 5 mL of the isolated and resuspended JM105 x pRscK60-del cells from the above-described shaken flask culture, 7 mg of tyrosol (final concentration in the batch 5.1 mM) and 5 mL of KPiE buffer. The second batch contained, likewise in a batch volume of 10 mL: 5 mL of the isolated and resuspended JM105 x pRscK60-del cells, 7 mg of tyrosol, 4.9 mL of KPiE buffer and 0.1 mL of 1 M sodium D-erythorbate x H₂O, dissolved in KPiE buffer (see HPLC test in example 2). The batches were incubated on a shaker (Infors) at 30° C. and 140 rpm. Samples from the test were taken after 0 h, 1 h, and 4 h, and analyzed by HPLC. The results are reported in table 3.

TABLE 3
Activity of isolated E. coli K12 JM105 × pRscK60-del
cells that express RscK60-del oxidase from the shaken flask
culture with regard to the biotransformation of tyrosol
to HTS without or in the presence of sodium D-erythorbate

| | Tyrosol [mM] | HTS [mM] | Tyrosol [mM] | HTS [mM] |
|---|---|---|---|---|
| | without | without | in the | in the |
| | addition | addition | presence of | presence of |
| Time | of sodium | of sodium | 10 mM sodium | 10 mM sodium |
| [h] | erythorbate | erythorbate | erythorbate | erythorbate |
| 0 | 5.1 | 0.0 | 5.1 | 0.0 |
| 1 | 1.6 | 1.7 | 0.8 | 4.5 |
| 4 | 0.0 | 2.1 | 0.0 | 5.2 |

### EXAMPLE 4

### Expression of RscK60-Del Oxidase in E. coli by Fermentation

For the fermentation, the E. coli JM105 x pRscK60-del strain was used. The fermentations were conducted in Biostat B fermenters (working volume 2 1) from Sartorius BBI Systems GmbH.

### Shaken Flask Preculture:

2 x 100 mL of LBamp medium in 1 1 baffled Erlenmeyer flasks were inoculated from an agar plate with the JM105 x pRscK60-del strain and incubated on an incubation shaker (Infors) at 30° C. and a speed of 120 rpm for 7-8 h up to a cell density OD₆₀₀ of 2/mL-4/mL.

### Prefermenter:

1.5 L of FM2 medium, supplemented with 40 g/L glucose and 100 mg/L ampicillin, was inoculated with 7.5 mL of the shaken flask preculture. The fermentation conditions were: temperature 30° C.: pH constant at 7.0 (automatic correction with 25% NH₄OH and 6.8 N HPO₄ as described below); foam control by automatic metering of 4% v/v Struktol J673 in H₂O (Schill & Seilacher): stirrer speed 450-1300 rpm: constant ventilation with compressed air sterilized by means of a sterile filter at 1.7 vvm (vvm: input of compressed air into the fermentation batch reported in liters of compressed air per liter of fermentation volume per minute): pO₂≥50%. The partial oxygen pressure pO₂ was controlled via the stirrer speed. After a fermentation time of 16 h, a cell density OD₆₀₀ of 45/mL-60/mL was achieved.

### Production Fermenter:

1.35 L of FM2 medium, pH 7.0, supplemented with 20 g/L glucose, 0.5 mM CuSO₄ x 5 H₂O and 100 mg/L ampicillin, was inoculated with 150 mL of prefermenter culture. The fermentation conditions were: temperature 30° C.: pH constant at 7.0 (automatic correction with 25% NH₄OH and 6.8 N H₃PO₄ as described below); foam control by automatic dosage of 4% v/v Struktol J673 in H₂O (Schill & Seilacher): stirrer speed 450-1300 rpm: constant ventilation at 1.7 vvm: pO₂≥50%. The partial oxygen pressure pO₂ was controlled via the stirrer speed. The fermentation time was 30-32 h.

FM2 medium: (NH₄)₂SO₄, 5 g/L: NaCl, 0.50 g/L: FeSO₄ x 7 H₂O, 0.075 g/L: Na₃ citrate, 1 g/L, MgSO₄ x 7 H₂O, 0.30 g/L, CaCl₂ x 2 H₂O, 0.015 g/L, KH₂PO₄, 1.50 g/L, vitamin B1 (Sigma-Aldrich), 0.005 g/L: peptone (Oxoid), 5.00 g/L: yeast extract (BD Biosciences), 2.50 g/L: trace element solution, 10 mL/L (corresponding to that used in example 2).

The pH in the fermenter was adjusted to 7.0 at the start by pumping in a 25% NH₄OH solution. During the fermentation, the pH was kept at a value of 7.0 by automatic correction with 25% NH₄OH, or 6.8 N H₃PO₄. For inoculation, 150 mL of prefermenter culture was pumped into the fermenter vessel. The starting volume was thus 1.5 L. At the start, the cultures were stirred at 350 rpm and sparged at a ventilation rate of 1.7 vvm. Under these starting conditions, the oxygen probe was calibrated to 100% saturation before the inoculation.

The target value for the O₂ saturation (pO₂) during the fermentation was adjusted to 50%. After the O₂ saturation had dropped below the target value, a closed-loop control cascade was started, in order to bring the O₂ saturation back to the target value. The stirrer speed was increased continuously (up to max. 1500 rpm).

The fermentation was conducted at a temperature of 30° C. Once the glucose content in the fermenter, from initially 20 g/L, had dropped to about 5 g/L, a 60% (w/w) glucose solution was fed in continuously. The feed rate was adjusted such that the glucose concentration in the fermenter never exceeded 2 g/L again thereafter. Glucose was determined with a glucose analyzer from YSI (Yellow Springs, Ohio, USA).

Once the target density in the fermenter had reached an OD₆₀₀ of 50/mL-60/mL (fermentation time 7.5 h), the expression of the oxidase RscK60-del was started by a single addition of the IPTG inductor (final concentration 0.4 mM). 22.5 h after induction, corresponding to a total fermentation time of 30 h, the fermentation was stopped and the enzyme activity (L-DOPA test) and the conversion of tyrosol to HTS on an analytical scale (HPLC test) were quantified in a sample of the fermentation batch as described in example 2. In both tests, the fermenter broth was used directly without further workup. The remaining fermenter broth was dispensed in 50 mL aliquots, frozen and stored at −20° C. for further experiments.

The specific enzyme activity of the fermenter broth without further workup with L-DOPA as enzyme substrate (L-DOPA test) was 28.4 mU/mg protein, using 1.6 mL of fermenter broth with a protein concentration of 5 mg/mL in the L-DOPA test.

In the HPLC test for examination of the enzyme activity in the presence of sodium D-erythorbate (example 2), 100 μl of fermenter broth (OD₆₀₀ 63.8/mL) was used in the HPLC test after a fermentation time of 30 h, such that the actual cell density in the 10 mL test batch was 0.64/mL. Samples from the test were taken after 0 h, 1 h, 2 h and 4 h, the content of tyrosol and HTS was analyzed by HPLC, and the percentage yield, i.e. the proportion of tyrosol used that was converted to HTS, was determined (percentage of HTS). After an incubation time of 4 h, 99.4% of the tyrosol used had been converted to HTS (see table 4).

TABLE 4
Percentage of HTS over the course of a biotransformation
of tyrosol to HTS with E. coli K12 JM105 × pRscK60-del
fermenter cells that express RscK60-del oxidase

| Time [h] | Proportion of HTS [%] |
|---|---|
| 0 | 0.7 |
| 1 | 82.6 |
| 2 | 95.0 |
| 4 | 99.4 |

In a further batch, the suitability of the fermenter cells of E. coli strain JM105 x pRscK60-del, which is important for economic viability of the process, for the biotransformation of tyrosol to HTS was examined with regard to the conversion of tyrosol in a maximum concentration in a minimum period of time (space-time yield).

An initial charge of 249 mg of tyrosol (final concentration 180 mM) and 389 mg of sodium D-erythorbate x H₂O (180 mM) in a 100 mL Erlenmeyer flask was dissolved in 1 mL of KPi buffer, and 9 mL fermenter broth of the RscK60-del oxidase-expressing JM105 x pRscK60-del cells was added without further workup, in order to start the reaction. The batch volume was 10 mL. Molar ratio of tyrosol to sodium D-erythorbate x H₂O was 1:1. The pH of the reaction actually measured in the batch was 6.7. The batch was incubated on a shaker (Infors) at 37° C. and 140 rpm. Sampling and analysis by HPLC (as described in example 2) were effected after 0 h, 2 h and 4 h. The progression of the reaction is shown in table 5.

TABLE 5
Progression against time of the biotransformation of tyrosol
to hydroxytyrosol (HTS) with RscK60-del fermenter cells

| Time [h] | Tyrosol [g/L] | HTS [g/L] |
|---|---|---|
| 0 | 24.9 | 0.0 |
| 2 | 10.2 | 14.0 |
| 4 | 0.0 | 25.5 |

### EXAMPLE 5

### Production of HTS

Experiment 1: Biotransformation of 30 g/L tyrosol with JM105 x pRscK60-del fermenter cells that express RscK60-del oxidase in the presence of sodium D-erythorbate x H₂O in a molar ratio of tyrosol to sodium D-erythorbate of 1:1

An initial charge of 300 mg of tyrosol (final concentration 220 mM) and 475.4 mg of sodium D-erythorbate x H₂O (220 mM) in a 100 mL Erlenmeyer flask was dissolved in 5 mL of KPIE buffer, and 5 mL of fermenter broth of the E. coli K12 JM105 x pRscK60-del strain from example 4 was added without further workup in order to start the reaction. The batch volume was 10 mL. The molar ratio of tyrosol to sodium D-erythorbate x H₂O was 1:1. The pH of the reaction actually measured in the batch was 6.7. The batch was incubated on a shaker (Infors) at 30° C. and 140 rpm. Sampling and analysis by HPLC (example 2) were effected after 0 h, 3 h and 24 h. The progression of the reaction is shown in table 6.

TABLE 6
Progression against time of the biotransformation of tyrosol
to HTS with E. coli K12 JM105 × pRscK60-del fermenter cells

| | Tyrosol | Tyrosol | HTS | HTS |
|---|---|---|---|---|
| Time [h] | [g/L] | [mM] | [g/L] | [mM] |
| 0 | 28.36 | 205.20 | 0.10 | 0.63 |
| 3 | 22.30 | 161.34 | 7.98 | 51.78 |
| 24 | 0.00 | 0.00 | 30.53 | 197.99 |

Experiment 2: Biotransformation of 60 g/L tyrosol with RscK60-del fermenter cells in the presence of sodium D-erythorbate x H₂O (molar ratio of tyrosol to sodium D-erythorbate 1:1.2)

An initial charge of 600 mg of tyrosol (final concentration 434 mM) and 1126 mg of sodium D-erythorbate x H₂O (521 mM) in a 100 mL Erlenmeyer flask was suspended in 5 mL of KPiE buffer. While sodium D-erythorbate x H₂O had good solubility under these conditions, tyrosol could be reacted only in suspension. 5 mL of fermenter broth of the E. coli K12 JM105 x pRscK60-del strain from example 4 was added without further workup in order to start the reaction. The batch volume was 10 mL. The molar ratio of tyrosol to sodium D-erythorbate x H₂O was 1:1.2. The batch was incubated on a shaker (Infors) at 30° C. and 140 rpm. Sampling and analysis by HPLC (example 2) were effected after 0 h, 3 h and 24 h. The progression of the reaction is shown in table 7.

TABLE 7
Progression against time of the biotransformation of tyrosol
to HTS with E. coli K12 JM105 × pRscK60-del fermenter cells

| | Tyrosol | Tyrosol | HTS | HTS |
|---|---|---|---|---|
| Time [h] | [g/L] | [mM] | [g/L] | [mM] |
| 0 | 60.0 | 434 | 0.1 | 1 |
| 3 | 50.6 | 366 | 8.6 | 56 |
| 24 | 0.0 | 0 | 66.7 | 433 |

Experiment 3: Biotransformation of 100 g/L tyrosol with RscK60-del fermenter cells in the presence of sodium D-erythorbate x H₂O (molar ratio of tyrosol to sodium D-erythorbate 1:1.2)

To an initial charge of 1000 mg of tyrosol (final concentration 724 mM) and 1885 mg of sodium D-erythorbate x H₂O (872 mM) in a 100 mL Erlenmeyer flask were added 1 mL of KP₂ buffer (500 mM potassium phosphate, 10 mM EDTA, pH 6.5) and 9 mL of fermenter broth of the cells JM105 x pRscK60 that express RscK60-del oxidase from example 4 without further workup. While sodium D-erythorbate x H₂O had good solubility under these conditions, tyrosol could be reacted only in suspension. The batch volume was 10 mL. The molar ratio of tyrosol and sodium D-erythorbate x H₂O was 1:1.2. The batch was incubated on a shaker (Infors) at 37° C. and 140 rpm. Sampling and analysis by HPLC (example 2) were effected after 3 h, 6 h, 24 h and 29 h. The progression of the biotransformation against time is shown in table 8.

TABLE 8
Progression against time of the biotransformation of tyrosol
to HTS with E. coli K12 JM105 × pRscK60-del fermenter cells

| | Tyrosol | Tyrosol | HTS | HTS |
|---|---|---|---|---|
| Time [h] | [g/L] | [mM] | [g/L] | [mM] |
| 3 | 104.2 | 750 | 14.6 | 90 |
| 6 | 108.3 | 780 | 34.5 | 220 |
| 24 | 17.5 | 130 | 85.9 | 560 |
| 29 | 6.1 | 40 | 109.0 | 707 |

### EXAMPLE 6

### Extraction of HTS

6.5 mL of the biotransformation from example 5 experiment 2 was extracted four times with 10 mL of ethyl acetate each time (ethyl ethanoate, CAS number 141-78-6) and, after phase separation, the upper ethyl acetate phases were removed and combined. The combined ethyl acetate phases (volume 40 mL) were analyzed by HPLC for the presence of tyrosol and HTS as described in example 2, but only HTS could be detected. On the basis of the HPLC analysis, the purity of the HTS was 97%.

The molar yield of HTS was calculated. The amount of the tyrosol reactant used was 390 mg (6.5 mL of the 60 g/L tyrosol batch), corresponding to 2.8 mmol of tyrosol (molecular weight of tyrosol: 138.2 g/mol). Given a 100% yield, the result was 2.8 mmol HTS, corresponding to 431.8 mg (molecular weight of HTS: 154.2 g/mol). Determination by HPLC found 404.8 mg of HTS, which corresponded to 93.8% of the theoretical yield.

The solvent of the extract was separated off in a rotary evaporator (Büchi Rotavapor R-205)(bath temperature 62° C., reduced pressure of 500 mbar) and residues of the solvent were removed by reducing the pressure. The remaining brownish-yellow oil was weighed. The yield was 400 mg, which was in good agreement with the yield determined by HPLC.

### EXAMPLE 7

### Production of HTS on a Preparative Scale

A jacketed 1 L thermostatable glass vessel (Diehm) was connected via a hose connection to a thermostat (Lauda) and adjusted to a temperature of 37° C. To an initial charge of 35 g of tyrosol (final concentration 507 mM) and 65.6 g of sodium D-erythorbate x H₂O (607 mM) in the glass vessel were added 250 mL of KP₃ buffer (50 mM potassium phosphate, 5 mM EDTA, pH 6.5) and 250 mL of fermenter broth of the E. coli K12 JM105 x pRscK60-del strain (example 4) without further workup. The molar ratio of tyrosol to sodium D-erythorbate x H₂O was 1:1.2. The batch volume was 0.5 L. The mixing was effected by means of a magnetic stirrer. For oxygen supply, compressed air was introduced into the batch by a glass tube. The reaction was started by starting the magnetic stirrer and the sparging. Sampling and analysis by HPLC (example 2) were effected after 0 h, 3 h, 6 h, and 24 h. At that time, the tyrosol had been 100% converted. The progression of the biotransformation against time is summarized in table 9.

TABLE 9
Biotransformation of tyrosol to HTS with RscK60-
del fermenter cells on a preparative scale

| | Tyrosol | Tyrosol | HTS | HTS |
|---|---|---|---|---|
| Time [h] | [g/L] | [mM] | [g/L] | [mM] |
| 0 | 70.0 | 506.5 | 0.0 | 0.0 |
| 3 | 46.9 | 339.2 | 17.1 | 110.6 |
| 6 | 26.5 | 191.8 | 41.0 | 266.1 |
| 24 | 0.0 | 0.0 | 70.6 | 458.0 |

The molar yield of 458 mM HTS was 90.4%, based on the amount of 506.5 mM tyrosol used at the start of the reaction.

For isolation of HTS, 0.5 L of the biotransformation was first incubated at 80° C. while mixing with a magnetic stirrer for 30 min and then centrifuged at 4000 rpm for 30 min (Heraeus Megafuge 1.0 R) in order to separate off particulate material. The supernatant was extracted three times with ethyl acetate. The first extraction was with 1 L of ethyl acetate, and the second and third extractions each with 0.5 L of ethyl acetate. The ethyl acetate phases were combined and gave 2 L of extract.

The ethyl acetate was distilled off in a rotary evaporator (Büchi Rotavapor R-205), first at a reduced pressure of 270 mbar and a temperature of 60° C. and then at a reduced pressure of 20 mbar and a temperature of 85° C., in order to remove residual ethyl acetate. Removal of the ethyl acetate left a residue of 34.1 g, which corresponded to the HTS product of the process. To determine the purity, 32 mg of the HTS product was weighed out, dissolved in 1 mL of H₂O (concentration 32 mg/mL) and analyzed by HPLC. The HPLC analysis gave an HTS content of 30 mg/mL), which corresponded to a purity of 93.8% based on the amount of 32 mg of the HTS product weighed out.

The yield of HTS was calculated. The amount of the tyrosol reactant used was 35 g. Taking account of the differences in molecular weight (138.2 g/mol for tyrosol, 154.2 g/mol for HTS), a maximum yield of 39 g of HTS was to be expected. Taking account of the purity of 93.8%, 34.1 g of the HTS product contained 31.9 g of HTS. For the overall process, this corresponded to a yield of 81.7%, based on the maximum achievable yield of 39 g of HTS.

1. A process for producing hydroxytyrosol (HTS) by enzymatic conversion of tyrosol to HTS, characterized in that the reaction mixture comprises
i) tyrosol and
ii) a compound selected from the group consisting of erythorbic acid and erythorbate and
iii) an oxidase with an amino acid sequence selected from the group consisting of SEQ ID NO: 2 and an amino acid sequence homologous with SEQ ID NO: 2 and
HTS is isolated from the reaction mixture.

2. The process as claimed in claim 1, characterized in that the amino acid sequence homologous with SEQ ID NO: 2 is SEQ ID NO: 3.

3. The process as claimed in either of claims 1 and 2, characterized in that the oxidase is produced by recombinant means by fermentation in E. coli.

4. The process as claimed in claim 3, characterized in that the fermentation for production of the oxidase is effected in the presence of a concentration of at least 0.02 mM Cu(II) ions.

5. The process as claimed in either of claims 3 and 4, characterized in that the fermenter broth from the fermentation for production of the oxidase is used directly without further workup in the process for producing HTS.

6. The process as claimed in any of claims 1 to 5, characterized in that tyrosol is used in a concentration of more than 200 mM.

7. The process as claimed in any of claims 1 to 6, characterized in that the reaction mixture contains erythorbic acid or erythorbate in a concentration of at least 0.4 M.

8. The process as claimed in any of claims 1 to 7, characterized in that the molar ratio of tyrosol to erythorbic acid or erythorbate is not more than 1:1.50.

9. The process as claimed in any of claims 1 to 8, characterized in that the proportion by volume of the fermenter broth from the fermentation for production of the oxidase in the reaction mixture is up to 90%.

10. The process as claimed in any of claims 1 to 9, characterized in that the reaction mixture is reacted for a period of time until at least 90% of the tyrosol used has been converted to HTS.

11. The process as claimed in any of claims 1 to 10, characterized in that HTS is produced from tyrosol with a molar yield of at least 70%.

12. The process as claimed in any of claims 1 to 11, characterized in that HTS is isolated from the reaction mixture by extraction with ethyl acetate.

13. The process as claimed in any of claims 1 to 12, characterized in that HTS is isolated from the reaction mixture with a purity of at least 80%.
